(19) United States
(12) Patent Application Publication
(10) Pub. No.: US 2017/0065789 A1
(43) Pub. Date: Mar. 9, 2017

(54) Respiratory Tee Piece

(71) Applicant: George Ashford Reed, Lake Oswego, OR (US)

(72) Inventor: George Ashford Reed, Lake Oswego, OR (US)

(73) Assignee: Medica Holdings LLC, Lake Oswego, OR (US)

(21) Appl. No.: 14/848,060

(22) Filed: Sep. 8, 2015

(51) Int. Cl. A61M 16/08; A61M 16/20

(52) U.S. Cl. CPC A61M 16/0833; A61M 16/208

(57) ABSTRACT

A Tee piece that functions as device to direct dual, alternating respiratory therapies to a patient. It has a centrally located low pressure actuated one way valve that connects the inhalation and exhalation paths to the specific therapy devices. The gravity hung valve has a low pressure activation and an offset, angled seat that allows the valve itself to be completely removed from the path of the medicated aerosol particles so as to minimize the potential for particle condensation by collision with the valve. The valve forms a holding chamber for the medicated aerosol increasing the efficiency of the nebulizer cycle. The physical design of the Tee piece's outside surface prevents reverse connection, which is a common problem and the oval shape of the PEP port eliminates blow-off of the PEP device by excessive back pressure.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF INVENTION

The present invention relates to a novel design for a respiratory Tee piece that is capable of connection to multiple respiratory therapy devices or medicated aerosol delivery units. It enables a new level of efficiency in both product delivery and device condensation losses for those who need such respiratory therapy treatments. It is adapted to matingly connect with two standardized respiratory devices simultaneously to allow for combination, enhanced, single step, respiratory treatments.

Respiratory treatments vary considerably as do the different manufacturer's devices used to facilitate the treatments. Generally, the patient undergoes separate regimens of inhalation and exhalation treatments. First, the exhalation treatments require the patient to exhale through any of a plethora of devices that send a resultant pulsation pressure wave back down into their lungs to dislodge phlegm. Once enough cycles have been performed, the patient's breathing ability is improved and they can then undergo an inhalation treatment wherein they receive a medicated aerosol (generally inhaled corticosteroids) to further increase their lung capacity and ease their labored breathing.

This type of respiratory therapy has two drawbacks. First, it is time consuming to perform these two exhalation and inhalation treatments. Second, the patient's, often older and frail, require breaks between treatments, further lengthening the time medical personal is required. Since time is money—this is not good.

Further, many of the devices that have tried to combine the two types of respiratory therapies have failed in maintaining a high percentage of the flow of the aerosol particles in the desirable 5-micron diameter. The aerosol particles are generally not constrained in the aerosol section of the device and are some are lost portion in the exhalation phase where they are swirled around in the device to collide and condense. Others escape because of the open inhalation end when the patient takes their mouth off of the device. Lastly, in devices that utilize a valve system, aerosol particles collide with any valve therein as it opens in the inhalation therapy phase. All three mechanisms reduce the amount of medicated aerosol particles that are delivered to the patient and increase the average size of the aerosol particles delivered.

Henceforth, an improved respiratory Tee piece that minimizes the time required for conventional respiratory treatments, allows the patient to remove their mouth momentarily from the device without significant aerosol losses, minimizes the amount of aerosol condensing on the devices inner walls, and maximizes the aerosol particle size transmission efficiency, would fulfill a long felt need in the respiratory treatment industry. This new invention utilizes and combines known and new technologies in a unique and novel configuration to overcome the aforementioned problems and accomplish this.

### SUMMARY OF THE INVENTION

In accordance with the invention, the objects of the present invention, which will be described subsequently in greater detail, is to provide an improved respiratory Tee piece to accommodate simultaneous exhalation and inhalation therapies.

It is a further object of this invention to provide a respiratory Tee piece capable of constraining a medical aerosol generated by a nebulizer for later release with minimal losses of aerosol particles.

It is a last object of this invention to provide a respiratory Tee piece that optimizes the percentage of aerosol particles delivered in the preferred size, minimizing the condensation losses on any attendant valves within the Tee piece, therein delivering more aerosol to the lungs.

The improved respiratory Tee piece has many of the advantages mentioned heretofore and many novel features that result in a new Tee piece which is not anticipated, rendered obvious, suggested, or even implied by any of the prior art, either alone or in any combination thereof.

The subject matter of the present invention is particularly pointed out and distinctly claimed in the concluding portion of this specification. However, both the organization and method of operation, together with further advantages and objects thereof, may best be understood by reference to the following description taken in connection with accompanying drawings wherein like reference characters refer to like elements. Other objects, features and aspects of the present invention are discussed in greater detail below.

### BRIEF DESCRIPTION OF THE DRAWINGS

FIG. 1 is a left side view of the respiratory Tee piece with the pressure activated valve shown withdrawn;

FIG. 2 is an aerosol delivery end view of the respiratory Tee piece with the pressure activated valve shown withdrawn;

FIG. 3 is a top view of the respiratory Tee piece;

FIG. 4 is a bottom view of the respiratory Tee piece;

FIG. 5 is a front end view of the respiratory Tee piece with the pressure activated valve shown withdrawn, and a section line A-A drawn vertically through the approximate center;

FIG. 6 is a side cross sectional view taken through section line A-A; and

FIG. 7 is a front view of the low pressure activated valve.

### DETAILED DESCRIPTION

The above description will enable any person skilled in the art to make and use this invention. It also sets forth the best modes for carrying out this invention. There are numerous variations and modifications thereof that will also remain readily apparent to others skilled in the art, now that the general principles of the present invention have been disclosed.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

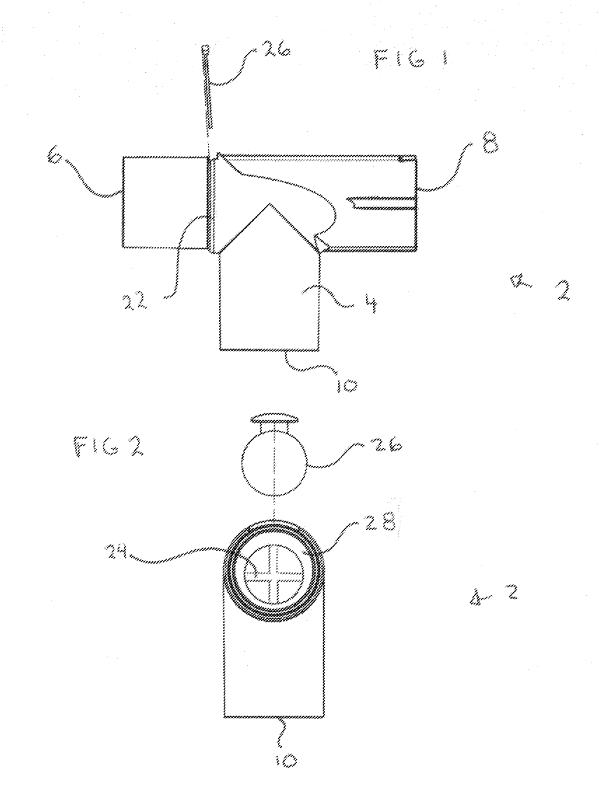

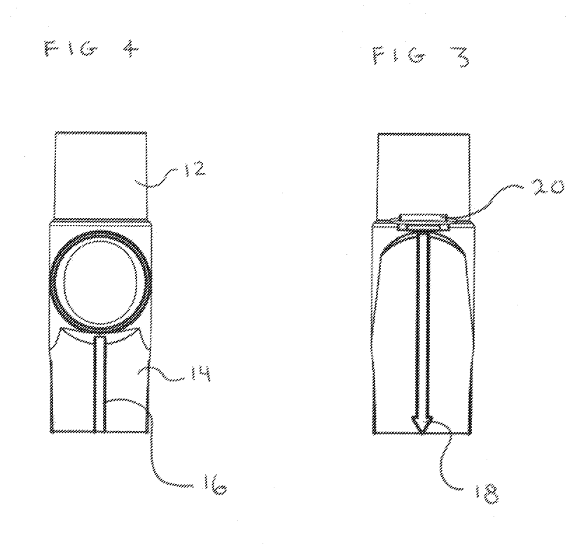

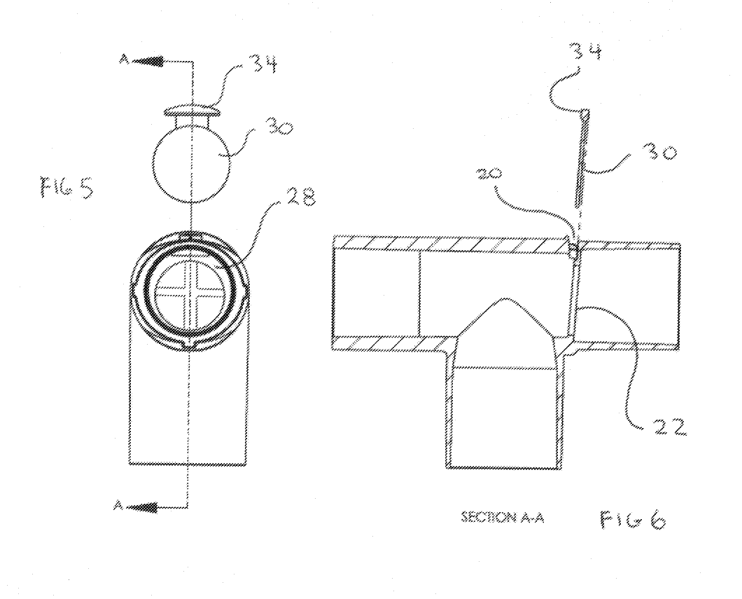

Looking at FIGS. 1 and 2 it can be seen that the aerosol Tee piece 2 has a Tee shaped body 4 with three generally circular port openings. The aerosol delivery port 6 and the patient port 8 reside centered about the same horizontal axis, and the positive exhalation pressure (PEP) port 10 resides centered about a vertical axis that intersects the horizontal axis at its approximate midpoint. Thus the centerline of the PEP port 10 resides at 90 degrees from the common centerline of the patient port 8 and the aerosol delivery port 6. The aerosol delivery port 6 is designed for connection to a medical aerosol generating device and is sized to the standard ISO 22 mm circular outer diameter for connection with the industry standard nebulizers and similar functioning devices. Its outer surface 12 is circular and unadorned. The patient port 8 is designed to accept a standardized replaceable mouthpiece (known in the art and not illustrated) and it has an ISO 18 mm circular inner diameter, however its outer surface 14 is oval and has a series of four horizontal linear ridges 16 equally arranged at 90 degree radial separation about the surface. (FIGS. 3 and 4) The uppermost, top ridge has an arrow 18 at its end pointing toward the patient. The combination of the oval exterior configuration, the horizontal ridges 16 and the arrow 18 are intended to present an obstacle and a visual reminder to ensure improper connections and use of the device 2.

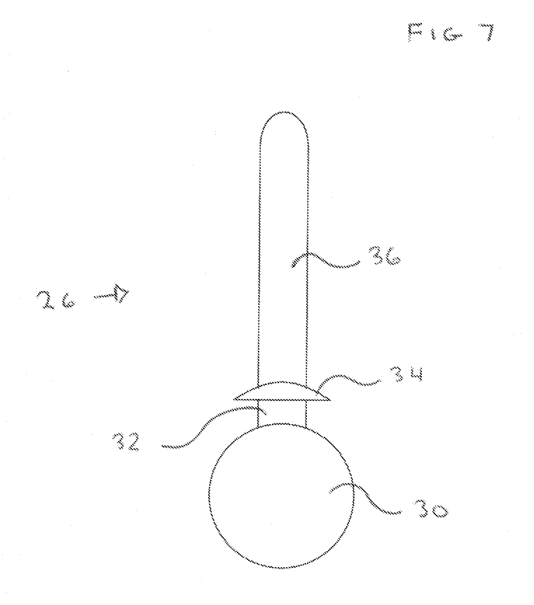

The body 4 has a flapper hinge horn slot 20 cut into its top surface that resides between the aerosol delivery port 6 and the PEP port 10. Between the horn slot 20 and the aerosol deliver port 6, extending normally from the circular inner wall of the body 4 is an angled flapper valve seat 22 that is suspended from a neck 32. (Best seen in FIGS. 2 and 6) The valve seat 22 is circular but its center point lies below the common horizontal axis that the aerosol delivery port 6 and the patient port 8 are centered about. Across the opening of the valve seat 22 is a excessive exhaust exhalation brace 24 to prevent the reverse movement of the flapper valve 26 beyond the sealing face 28 of the valve seat 22 (FIG. 5) towards the aerosol delivery port 6. Although depicted as an “X” brace it may have other configurations as is well known in the industry.

The final component of the tee piece 2 is the flapper valve 26 as illustrated best in FIG. 7. The flapper valve 26 has a valve flap 30, a neck 32, a hinge horn 34 and an installation tab 36. The valve flap 30 is gravity hung by the frictional engagement of the hinge horn 34 in the flapper hinge horn slot 20. The neck 32 is the hinge mechanism that the valve flap 30 swings on. The neck 32 allows the valve flap 30 to gravity hang into the valve body with enough spatial clearance so that no part of the valve flap 30 touches any art of the interior wall of the Tee piece's body. In this way the valve flap 30 is a very low pressure actuated valve. The neck 32 defines a length between the hinge horn 34 and the valve flap 30 that is less than the distance that the offset valve seat 22 extends form the interior wall of the Tee piece body 4. The opening pressure is dependent on the weight of the valve flap 30 itself. The valve flap 30 has a thickness that is less than the height that the valve flange extends from the interior wall of the Tee piece body 4 This design allows for the valve flap 30 to swing completely horizontal by the neck 32 so that it resides behind the valve seat and out of the way of the incoming medicated aerosol during the inhalation cycle. In the preferred embodiment the valve flap 30 is made of medical silicon and its thickness is selected to be the minimum adequate to prevent distortion and allow the valve flap 30 to remain planar to make a proper seal through its service life.

The flapper valve 26 is replaceable if the need arises. To install the flapper valve 26 is fed horizontally through the patient port 8 and the detachable installation tab 36 is fed up through the flapper hinge horn slot 20 until the hinge horn 34 contacts the top of the Tee piece's upper inner wall and the flapper hinge slot 20. The hinge horn 34 is dimensionally wider than the inside opening of the horn slot 20 and will not pass. The installation tab 36, now on the outside of the Tee piece 2 is rocked side to side as tension away from the Tee piece 2 is applied so as to pull each side of the horn up through the inside opening of the horn slot 20. (The flapper valve is elastically deformable and in the preferred embodiment is made of a medical grade silicon.) The outside opening of the horn slot 20 is larger than the inside opening and is sized to retain the hinge horn 34 within it such that when the installation tab 36 is cut of at the top of the hinge horn 34, the hinge horn 34 will remain flush with the outer surface of the Tee piece 2. The installation tab 36 is now detached and valve flap 30 now remains hung by its neck 32 which forms the single suspension point that would allow it to freely hang by gravity into the interior cavity of the Tee piece 2 such that its planar face resides vertical with respect to the longitudinal axis that passes through the midpoint of the aerosol delivery port 6 and the patient port 8.

As can be seen in FIGS. 1 and 6 the flapper valve seat 22 is angled with its bottom toward the patient port 8. With the bottom of the seat 22 angled forward slightly from the suspension point of the valve flap 30 (the horn slot 20) it allows the circular peripheral edge of the valve flap 30 to contact the circular offset flange of the valve seat 22 enabling a one way, gravity operated seal that is closed upon exhalation pressure from the patient port 8 and opened upon inhalation pressure from the patient port. The excessive exhalation brace 24 prevents excessive exhalation pressure from forcing the valve flap 30 beyond the patient port side of the valve seat 22.

The valve seat 22 is offset horizontally from the common linear axis that the aerosol delivery port 6 and the patient port 8 are centered about. This allows room for the valve flap 30 to swing open fully upward to contact the upper inner wall of the Tee piece 2 so as to be behind the valve seat 22 and out of the flow of the aerosol particles traversing from the aerosol delivery port 6 to the patient port 8, thereby minimizing any particle collision condensation.

In operation, the Tee piece (with its one way integrated valve) when connected at its aerosol delivery port 6 to a medical aerosol nebulizer and at its PEP port to a positive exhalation pressure respiratory therapy device, will allow the patient to receive alternating cycles of PEP pressure to loosen the phlegm in their lungs followed by medicated aerosol inhalation. The combination of which is more effective and a faster way to provide respiratory therapy to a patient. The tapered oval configuration of the PEP port 10 allows for an extra secure connection with the PEP device which is important because there is a backpressure developed in its use so that the PEP device sees a force trying to dislodge it from the Tee piece 2. The four horizontal linear ridges 16 keep the patient from using the wrong end or hooking up the Tee piece 2 in reverse. When the patient stops inhaling the valve flap 32 swings closed by the effect of gravity (gravity closed valve.) When the patient exhales, the valve flap 30 is forced into an even tighter seal with the valve seat 22 and the patients lung air is directed down through the PEP port 10 wherein an oscillatory pulsation wave of air pressure is sent down the patients lungs. During this time any medicated aerosol that is generated by the nebulizer is built up and contained in the closed off section of the Tee piece 2 between the valve flap 30 and the aerosol port 6. The patient may now inhale such that the valve flap 30 will open, out of the path of the aerosol particle stream allowing a does of medicine into their lungs. The valve provides three methods of minimizing losses. First, it opens completely out of the aerosol pathway because of its minimized weight and the offset valve seat 22. Second with the valve closed on the exhalation cycle, it prevents any of the aerosol from swirling with the exhaled air and escaping through the PEP device. Third, with the valve normally closed by gravity and the angled valve seat 22, the patient can take his mouth off of the Tee piece 2 to spit phlegm without the medicated aerosol escaping from the Tee piece 2 because the aerosol is contained between the valve and the aerosol delivery port 6. The Tee piece 2 allows the operation of a dual cycle respiratory therapy without degrading the size of the medicated aerosol particles of reducing the size of the medicated dose. (5 microns is the desired mean medicated aerosol particle size.) Additionally, studies have shown that use of a holding chamber or spacer, can increase the medicated aerosol delivery by as much as 36%. Thus optimal performance of the nebulizer is enhanced by the holding chamber created between the closed valve and the medicated aerosol port 6 that collects the non pressurized plume of aerosol medicine and disperses it to the patient.

Those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Having thus described the invention, what is claimed as new and desired to be secured by Letters Patent is as follows:

1. A device for respiratory therapy comprising:
A Tee shaped body having three ends, a first port, a second port and a third port each at one of said ends thereof;
a gravity closed, one way valve operationally disposed within said Tee shaped body, said valve having an open side and a closed side, wherein said second port and said third port ports are on said open side and said first port is on said closed side.

2. The device for respiratory therapy of claim 1 wherein said valve has a flapper valve insert and a valve seat sized for gravitational sealing engagement with said flapper valve insert.

3. The device for respiratory therapy of claim 2 wherein said first port and said second port have an opening centered about a common linear first axis and said third port has an opening centered about a second linear axis, and wherein said first and said second linear axes intersect.

4. The device for respiratory therapy of claim 3 wherein said valve seat extends inward from an interior surface of said Tee shaped body, and has a center that is offset from said first linear axis.

5. The device for respiratory therapy of claim 3 wherein said valve seat resides at an angle with respect to the said linear first axis.

6. The device for respiratory therapy of claim 4 wherein said valve seat resides at an angle with respect to the said linear first axis.

7. The device for respiratory therapy of claim 2 wherein said flapper valve insert has a valve flap connected by a hinge mechanism to a horn, said valve flap suspended by said horn in a slot formed through said Tee shaped body.

8. The device for respiratory therapy of claim 7 wherein said valve seat has a circular opening therethrough and is a planar ring having a height between said interior surface of said Tee shaped body and said valve seat's circular opening, and wherein said valve flap has a thickness that is less than said height of said valve seat.

9. The device for respiratory therapy of claim 8 wherein said third port has an oval exterior configuration.

10. The device for respiratory therapy of claim 9 wherein the exterior of said Tee shaped body adjacent to said second port has a series of raised detents formed thereon.

11. The device for respiratory therapy of claim 10 wherein said valve seat has a cross brace formed there across its circular opening.

12. The device for respiratory therapy of claim 11 wherein said valve flapper valve insert has a detachable installation tail extending from said horn.

13. The device for respiratory therapy of claim 8 wherein said hinge mechanism has a length between said horn and said valve flap that is less than said height of said valve seat.
